(19) United States
(12) Patent Application Publication
(10) Pub. No.: US 2009/0143868 A1
(43) Pub. Date: Jun. 4, 2009

(54) VACUUM BASED IMPRESSION AND ALIGNMENT DEVICE AND METHOD

(75) Inventors: Hector Casanova, Naperville, FL (US); Yeongchi Wu, Darien, IL (US)

Correspondence Address: ARENT FOX LLP, 1050 CONNECTICUT AVENUE, N.W., SUITE 400, WASHINGTON, DC, 20036, US

(73) Assignee: Center for International Rehabilitation, Chicago, IL (US)

(21) Appl. No.: 12/323,822

(22) Filed: Nov. 26, 2008

Related U.S. Application Data
(60) Provisional application No. 60/996,606, filed on Nov. 27, 2007.

(51) Int. Cl. A61F 2/80

(52) U.S. Cl. 623/34; 623/33; 623/36

(57) ABSTRACT

A malleable bag connected to a one-way valve can be used to provide a prosthesis with an accurate impression of a residual limb and to perfect accurate static and dynamic alignment of the prosthesis on a patient. Methods and systems allow to rapidly and accurately capture an impression of a residual limb, including soft tissue compression due to the weight of the patient.

This application claims priority from U.S. Provisional Patent Application No. 60/996,606, filed in the US Patent Office on Nov. 27, 2008, which is incorporated herein by reference in its entirety.

### BACKGROUND OF THE INVENTION

1. Field of Invention

Aspects of the present invention relate to a method and system for accurately capturing and providing an impression and alignment of a device. More specifically, aspects of the current invention relate to a method and system for capturing and providing an impression of a residual limb, and the static and dynamic alignment of a prosthesis.

2. Description of Related Art

Currently, millions of people around the world have various types of disabilities that require the use of orthopedic or prosthetic devices to improve their mobility and overall quality of life. A common type of prosthetic device is an artificial limb. Various techniques exist to fit an individual with an orthopedic device, such as an artificial arm or leg. For example, a plaster bandage can be used to create a negative mold of a limb. The negative mold can be filled with a material, such as plaster, which takes the shape of the limb and thus forms a positive mold. The positive mold can then be used to form a prosthetic socket that fits and secures the prosthetic device to the limb.

Prosthetic limb production is generally a complicated process. Conventionally, prosthetic limb production starts with casting a negative mold of the residual limb using, for example, plaster-of-Paris casting bandages. The negative mold is then filled with plaster-of-Paris to form a positive model, which is then modified according to the patient's anatomical measurements. Finally, a soft insert is fabricated over the model, followed by lamination with a polyester or acrylic resin, or vacuum forming with a thermoplastic, such as as polypropylene, to produce the prosthetic socket. The prosthetic socket is then joined with other modular components and aligned.

Other prosthetic systems exist that apply the principle of dilatancy to actually produce transtibial prosthesis or other limb prosthesis without the need for plaster-of-Paris. Recyclable materials may be used for this purpose, such as is involved in the dilatancy casting system, which uses sand to replace plaster-of-Paris for forming a negative mold and positive model. Also, low-cost, portable equipment for alignment and forming of transtibial prosthesis is generally used that can be transported and maintained, to reduce initial set up and maintenance costs. Such an approach is typically more simplified than using plaster-of-Paris, improves accuracy, minimizes technical errors, and provides more rapid formation of a negative mold of a patient's residual limb over plaster-of-Paris. The negative mold can be converted into a positive replica of the residual limb, and from this positive model, a prosthetic socket can be made.

However, among other things, none of the conventional techniques provide methods and systems for taking into account soft tissue pressure of the residual limb of a patient when the patient is provided with a mold for a prosthesis. Furthermore, none of the conventional techniques provide methods and systems to perfect or otherwise sufficiently address both static and dynamic alignment of the prosthesis, along with increased comfort of the patient.

### SUMMARY OF THE INVENTION

In light of the above described problems and unmet needs, various aspects of the current invention provide methods and systems for vacuum-based impression and alignment that utilize the dilatancy principle of a malleable bag to replicate the shape of, for example, a transtibial residual limb. According to various aspects of the current invention, a malleable bag connected to a one-way valve can be used to provide a prosthesis with an accurate impression of a residual limb and to perfect accurate static and dynamic alignment of the prosthesis on a patient. According to various aspects of the current invention, methods and systems allow rapid and accurate capture an impression of a residual limb, including soft tissue compression due to the weight of the patient. For example, a malleable recipient such as a malleable bag with adequate elastic properties may be used to capture the shape of the residual limb and to encapsulate, for example, the femoral condyles for suspension purposes.

According to various aspects of the current invention, the vacuum-sealed malleable bag, for example, may include a solid connector and a one-way valve that prevents air from filtering in of the vacuum-sealed environment. The one-way valve may be used both to inject air into the malleable bag and to remove air from it. According to various aspects of the current invention, injecting air into the malleable bag renders the malleable bag more malleable, and removing air from the malleable bag hardens the malleable bag. For example, a one-way valve may be inverted to permit the injection of air in the malleable bag to facilitate the placement of the malleable bag on the residual limb of the patient without distorting soft tissues, and may be reversed to remove air from the malleable such as, for example, a polyurethane gel bag to harden the bag. According to various aspects of the current invention, air may be evacuated from the gel bag via a manual pump operated autonomously by a person, or via a person using their lungs to suck air out of the gel bag via a tube, for example. In either case, the system according to aspects of the current invention does not require a vacuum or negative air pressure to be applied continuously while manufacturing a prosthesis.

Once an accurate impression of a patient's residual limb has been captured by the gel bag, static and dynamic alignment may be performed. Static alignment can include, for example, the alignment of the prosthesis as the patient is wearing the prosthesis, including while standing up and remaining still, and includes height adjustment. Dynamic alignment includes the alignment of the prosthesis as the patient is wearing the prosthesis, including walking with the prosthesis. The soft and malleable nature of the gel bag, combined with the vacuum-based impression, provides increased comfort for the patient.

According to various aspects of the current invention, once the gel bag is formed and hardened, a mold, which is a reverse impression of the gel bag and corresponds to an impression of the patient's limb, is formed. A copy of the residual limb is thus manufactured. It should be noted that, in some variations, the resulting shape and alignment data of the patient could be digitized and stored electronically. The mold may be placed on a vertical fabrication jig to allow for the production of a prosthetic limb that takes into account the individual and specific features of the residual limb of the patient, the patient's height and alignment as well as soft tissue compression due to the pressure of the patient's weight on the prosthesis as sensed and recorded during static and dynamic alignment.

According to various aspects of the current invention, the prosthesis may be manufactured by positioning the positive plaster-of Paris mold on the vertical jig and by draping a soft sheet of thermoplastic over the mold, the attachment features and an ankle block. The thermoplastic sheet may be softened prior to draping by heating in an over at a temperature of about 375° F. Once the thermoplastic is draped around the mold, the attachment features and the ankle block, the thermoplastic may be sealed around these features, and a vacuum may be applied inside the draped thermoplastic to remove air. As a result, the thermoplastic liner espouses shape of the mold and the ankle block, and of the space in between. Thus, a concave or X-shaped prosthesis is formed between the mold and the ankle block upon solidification of the thermoplastic liner and removal of the mold from the thermoplastic liner.

Advantages of some variations of the current invention include increasing the degree of comfort of the patient when wearing the prosthesis. Also, no vacuum pump is necessary during the manufacturing process to provide a continuous source of negative pressure, which allows the manufacturing of the prosthesis in remote locations, for example. Other advantages includes providing even compression of the soft tissues to create a total surface bearing configuration, maintaining consistency in quality of the manufactured prostheses, achieving static and dynamic alignment, and providing the ability for the device to be portable and does not require any electrical power. Other advantages according to various aspects of the current invention include simplifying traditional manufacturing techniques associated with monolithic design of prosthetic limbs, eliminating of the need for an open ended socket, allowing a larger degree of rotation and torque absorption during the stance phase of gait in the transverse plane to improve the level of comfort, improving function and stability while walking on an uneven terrain, reducing cost, increasing area of support between the ankle block and the prosthetic foot to reduce the stress on the prosthetic limb, and increasing suspension ability.

Additional advantages and novel features of these aspects of the invention will be set forth in part in the description that follows, and in part will become more apparent to those skilled in the art upon examination of the following or upon learning by practice of the invention.

### BRIEF DESCRIPTION OF THE DRAWINGS

Various exemplary aspects of the systems and methods will be described in detail, with reference to the following figures, wherein:

FIG. 1 is an illustration of a vacuum-based impression and alignment device according to various aspects of the current invention;

FIG. 2 is a more detailed illustration of a connector according to various aspects of the current invention;

FIG. 3 is a flow chart illustrating a method of manufacturing a prosthetic system according to various aspects of the current invention;

FIG. 4 is an illustration of a residual limb coupled to the vacuum-based impression and alignment device according to various aspects of the current invention;

FIG. 5 is an illustration of a vacuum-based impression and alignment system in operation on a patient according to various aspects of the current invention;

FIG. 6 is an illustration of a vertical jig with a positive mold according to various aspects of the current invention;

FIG. 7 is an illustration of a prosthetic system manufacturing step according to various aspects of the current invention;

FIG. 8 is an illustration of another prosthetic system manufacturing step according to various aspects of the current invention;

FIG. 9 is an illustration of a liner according to various aspects of the current invention;

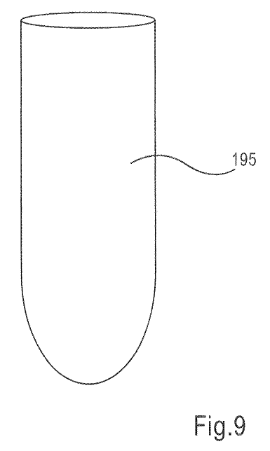

FIG. 10 is an illustration of another prosthetic system manufacturing step according to various aspects of the current invention;

FIG. 11 is an illustration of a prosthetic device according to various aspects of the current invention;

FIG. 12 presents an exemplary system diagram of various hardware components and other features, for use in accordance with an aspect of the present invention; and

FIG. 13 is a block diagram of various exemplary system components, in accordance with an aspect of the present invention.

### DETAILED DESCRIPTION OF PREFERRED ASPECTS

These and other features and advantages of this invention are described in, or are apparent from, the following detailed description of various exemplary aspects.

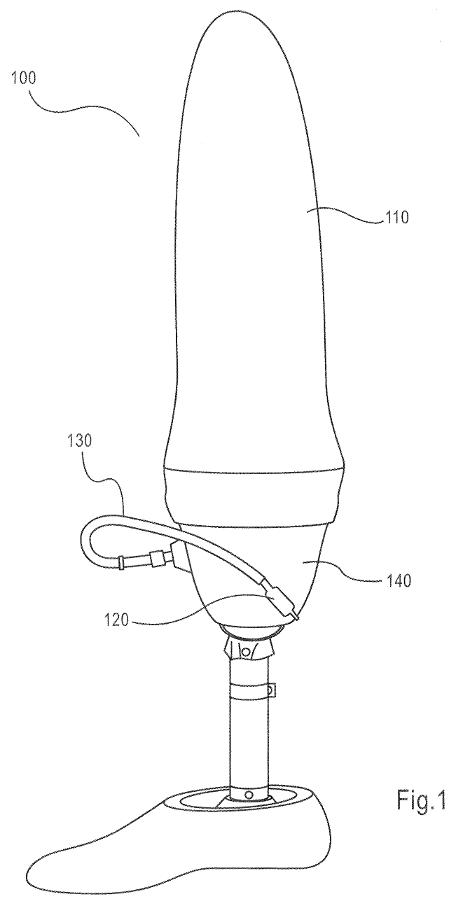

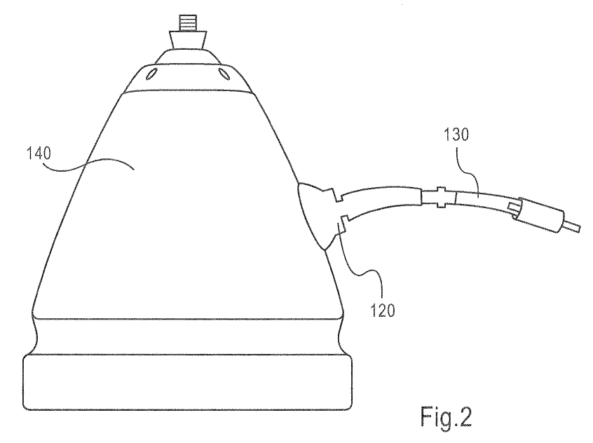

FIG. 1 is an illustration of an exemplary vacuum-based impression and alignment device according to various aspects of the current invention. In FIG. 1, the malleable bag such 110 such as a gel bag is connected to the solid connector 140, and may be coupled to the one-way valve 120 via the connector 140. The gel bag 110 may be coupled to the one-way valve 120 to prevent air from filtering out of the vacuum-sealed environment or to inject air into the gel bag 110, for example. The one-way valve 120 may be inverted to permit the removal of air from the gel bag 110 to, for example, harden the gel bag 110 once a residual limb is adjusted against the gel bag 110 in order to capture the shape of the residual limb. The one-way valve 120 may also be used to facilitate the placement of the gel bag 110 on the residual limb of a patient without distorting soft tissues (e.g., by reducing pressure therein). According to various aspects of the current invention, air may be evacuated from the gel bag 110 via a hand pump, a bicycle pump, or by a person using their lungs to suck air out of the gel bag 110 via the tube 130, for example. A negative pressure of about 20 inches Hg may generate enough pressure to harden the gel bag 110 for use, for example to create a solid negative impression of the residual limb of the patient before proceeding to static and dynamic alignment. FIG. 2 is a more detailed illustration of an exemplary connector 140 with the one-way valve 120 and the tube 130, according to various aspects of the current invention.

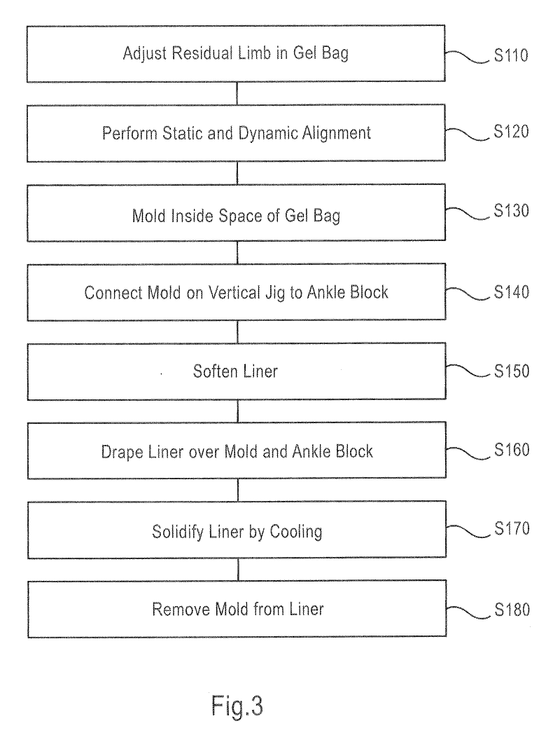

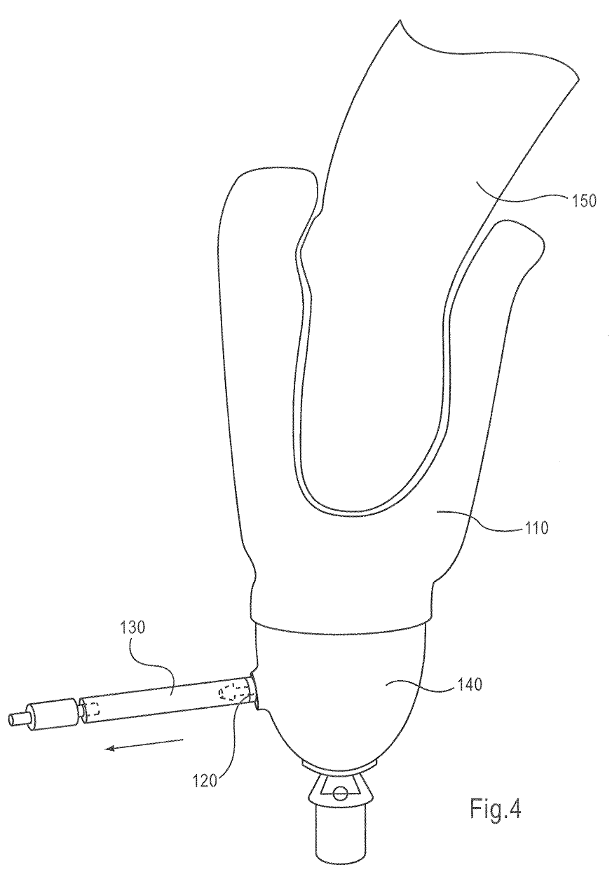

FIG. 3 is a flow chart illustrating a method of manufacturing a vacuum-based alignment system according to various aspects of the current invention. In FIG. 3, the method starts in S110, where a residual limb is adjusted in a gel bag. The residual limb, such as the residual limb 150 illustrated in FIG. 4, is inserted in a malleable recipient such as, for example, the gel bag 110. The dilatancy of the gel bag 110 is used, for example, to replicate the shape of a transtibial residual limb. The residual limb 150 is inserted in the gel bag 110 in such a way as to fit comfortably inside the gel bag 110, so as to allow a later molding of the precise imprint of the residual limb 150. Imprinting the residual limb 150 in the gel bag 110 can be performed due to the malleable nature of the gel bag 110, which may include polystyrene or other similar material that exhibits adequate elastic properties and/or hardening properties, to capture the shape of the residual limb 150 and to encapsulate, for example, the femoral condyle for suspension purposes. According to various aspects of the current invention, injecting air into the gel bag 110 renders the gel bag 110 more malleable, and removing air from the gel bag 110 hardens the gel bag 110. Thus, during S110, while the residual limb 150 is imprinted in the gel bag 110, air may be injected inside the gel bag 110 via a one-way valve, such as the valve 120 illustrated in FIG. 4, to mollify the gel bag 110 during the adjustment of the residual limb 150. Subsequently, once the residual limb 150 has been properly imprinted in the gel bag 110, air may be removed from the gel bag 110, as illustrated by the arrow in FIG. 4. When the air is removed from the gel bag 110 in S110, the gel bag 110 may be sufficiently hard to allow performing alignment of the residual limb 150 in the gel bag 110.

FIG. 4 is an illustration of the residual limb 150 partially inserted within an exemplary gel bag 110 in a vacuum-based impression and alignment system according to various aspects of the current invention. In FIG. 4, the residual limb 150 is pressed against the gel bag 110 until the gel bag 110 espouses the shape of the residual limb 150, including the effect of the pressure of the soft tissues of the residual limb 150. According to various aspects of the current invention, air may be injected in the gel bag 110 as the residual limb 150 is fitted within the gel bag 110 to allow a comfortable fit. However, once the residual limb 150 is properly fitted within the gel bag 110, air may be removed from the gel bag 110, as shown with the arrow pointing away from the connector 140 in FIG. 4, to harden the gel bag 110.

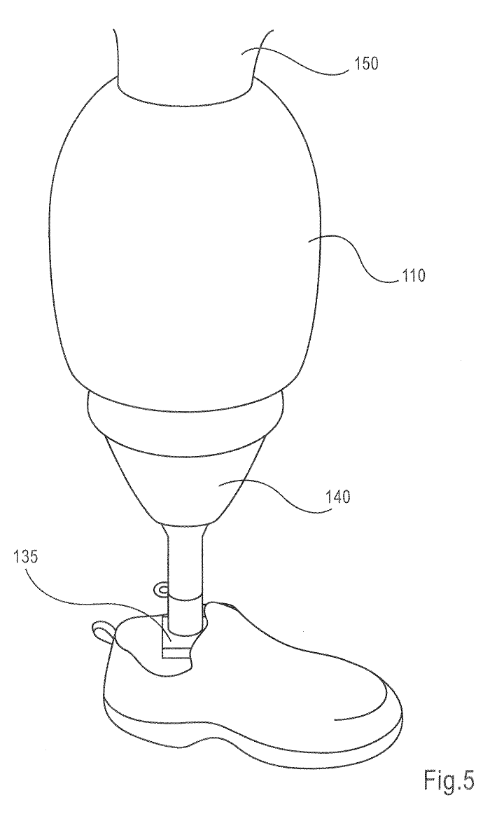

As shown in FIG. 3, during S120, alignment is performed. FIG. 5 is an illustration of an exemplary vacuum-based impression and alignment device according to various aspects of the current invention. The gel bag 110 may be connected to a simulated foot 145 via the connector 140 to provide a support for an accurate alignment of the residual limb 150 in the hardened gel bag 110. According to various aspects of the current invention, alignment includes static alignment and dynamic alignment. Static alignment may include alignment occurring when the patient is standing up on the gel bag 110, as illustrated in FIG. 5. It should be noted that air may be injected into the gel bag 110 so as to regain some malleability, such that the pressure of the soft tissues of the residual limb 150 on the gel bag 110 due to the weight of the patient may be recorded by the imprint formed on the gel bag 110. This approach simulates and reproduces a real life standing situation. As a result, a more precise imprint of the residual limb 150 may be formed on the gel bag 110. Static alignment may also include height adjustments, where a length of the residual limb 150 is taken into account in order to provide a proper equilibrium between both legs of the patient.

Dynamic alignment may include alignment occurring when the patient walking while the patient's residual limb 150 is in the gel bag 110. By injecting a small amount of air the gel bag 110 may become malleable to alter the imprint of the residual limb 150 and alignment under the increased pressure of the soft tissue of the residual limb 150 due to the weight and the gait of the patient. The soft and malleable nature of the gel bag, combined with the vacuum impression, provides increased comfort for the patient and increased accuracy in the capture of the imprint of the residual limb 150 and alignment. Additionally, suspension adjustments may be performed to ensure that the gel bag 110 is solidly attached to the patient's residual limb 150.

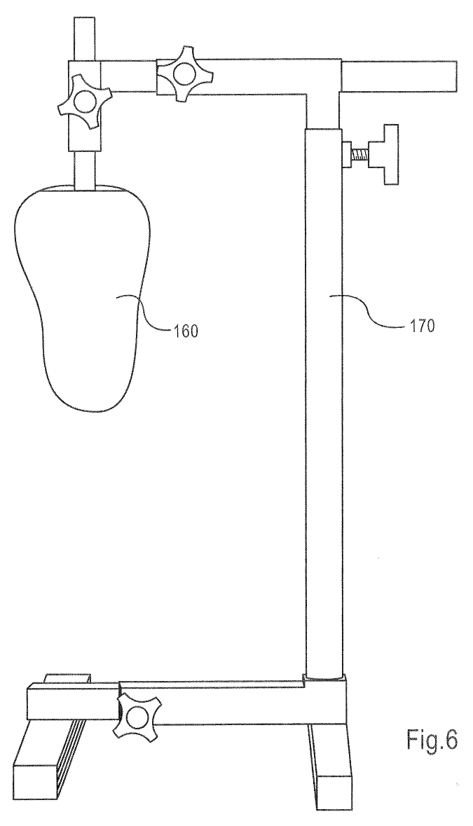

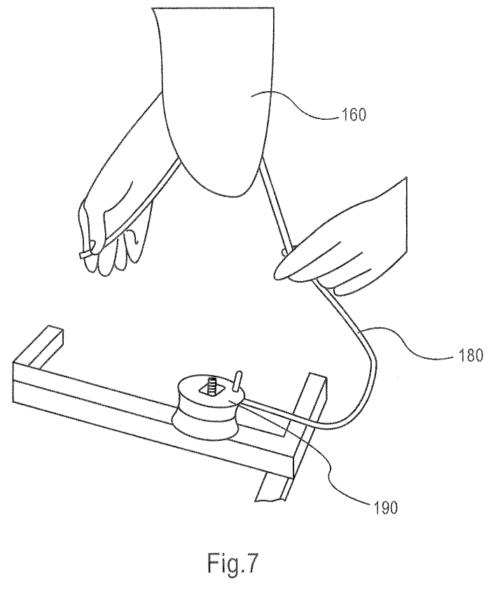

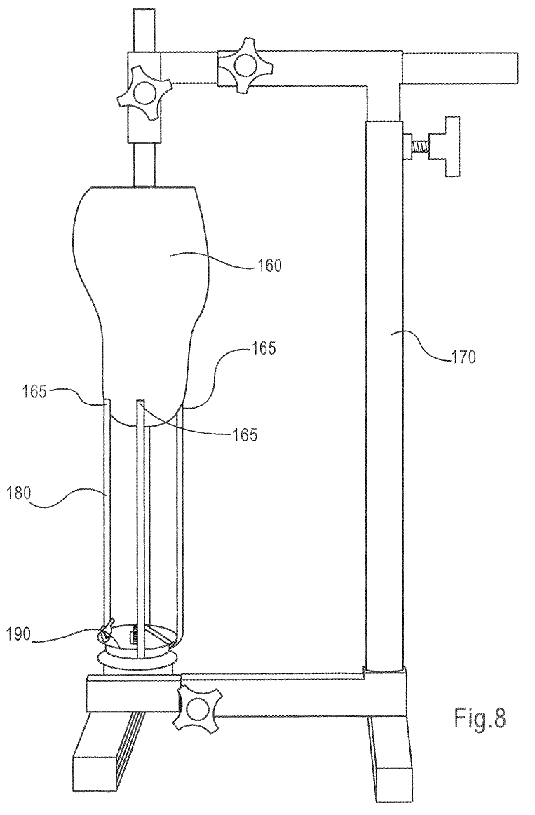

During S130 of FIG. 3, according to various aspects of the current invention, once static and dynamic alignment of the residual limb are achieved, a mold of the inside of the gel bag, which corresponds to a copy of the patient's residual limb, is formed. To that end, plaster may be used to form the mold 160, as illustrated in FIG. 6 (see limb 150, gel bag 110). The method continues to S140 in FIG. 3, where the mold is placed on a vertical jig. As shown in FIGS. 7-8, according to various aspects of the current invention, the mold 160 may be placed on the vertical jig 170 and connected via connection points 165 to an ankle block 190, for example, located vertically at a bottom portion of the vertical jig 170 via connectors 180. The resulting structure, as illustrated in FIG. 8, has the mold 160 tightly coupled to the ankle block 190 via connectors or securing features 180 (e.g., strings or cables). According to various aspects of the current invention, the connection points 165 may be elected to provide structural strength to the prosthesis and an accurate shape of the prosthesis taking into consideration the line-of-progression previously captured during dynamic alignment. Also, although elements 180 are illustrated as strings or cables, any other structure capable of connecting the mold 160 to the ankle block 190 via the attachment points 165 can similarly be used.

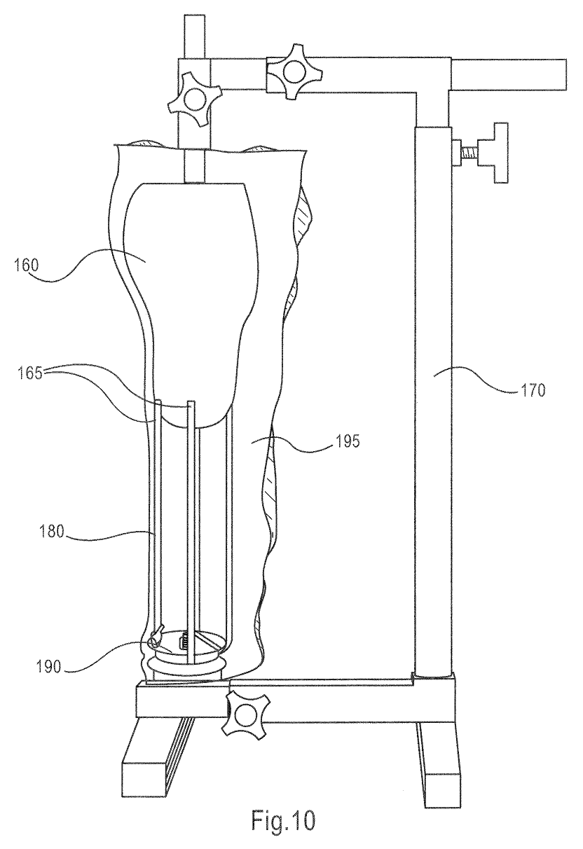

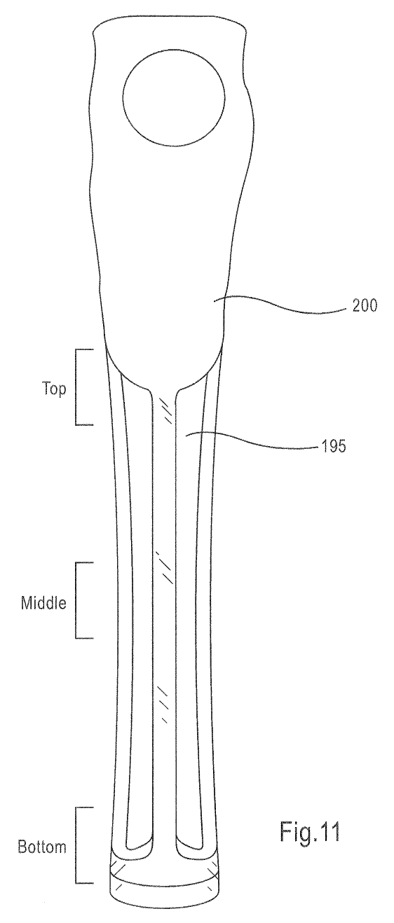

Next, as shown in FIG. 3, a thermoplastic sheet is softened in S150. According to various aspects of the current invention, the thermoplastic sheet may be softened by heating in an over at a temperature of about 375° F. Once the sheet is softened, the method continues to S160, where the sheet is draped over the mold, the attachment features and the ankle block. Also, a vacuum will be applied inside the draped softened thermoplastic sheet to remove air. FIG. 10 is an illustration of the thermoplastic sheet 195 draped and vacuum-formed over the mold 160, the connectors 180 and the ankle block 190. As a result, the thermoplastic sheet espouses the shape of the mold and the attachment features, and of the space in between. Thus, the liner has a concave or X shape. Next, the method of FIG. 3 continues to S170, where thermoplastic sheet or liner is left to dry and to solidify. Once the sheet has solidified, the method of FIG. 3 continues to S180, where the mold is removed from the solidified liner. FIG. 11 is an illustration of a solidified thermoplastic sheet 195, as part of the prosthesis 200, after the mold has been removed. The prosthesis 200 may have a concave or X shape, where the top and bottom portions of the prosthesis 200 are wider than the middle portion. According to various aspects of the current invention, the resulting structure with the solidified liner is a prosthesis that fits the patient's residual limb comfortably.

Additionally, once the prosthesis is produced, suspension adjustments can be performed to ensure that the prosthesis is solidly attached to the patient's residual limb. According to various aspects of the current invention, proper suspension can be achieved by using, for example, a neoprene sleeve or other similar material, to maintain the prosthesis properly connected to the residual limb of the patient.

Advantages of the current invention may include increased comfort of the patient when wearing the prosthesis, reduced need for a vacuum pump to provide a continuous source of negative pressure to the system during the manufacturing pressure, which allows the manufacturing of the prosthesis in remote locations, for example, capability for even compression of the soft tissues to create a total surface bearing configuration, increased consistency in the quality of manufactured prostheses, improved static and dynamic alignment, and enhanced portability of the device without requiring a separate power source. Other advantages according to various aspects of the current invention may include simplifying traditional manufacturing techniques associated with monolithic design of prosthetic limbs, eliminating of the need for an open ended socket, allowing rotation of up to 15°-20°, and absorbing torque during the stance phase of gait of the patient in the transverse plane, so as to improve the level of comfort, improving function and stability while walking on an uneven terrain, lowering cost, increasing area of support between the ankle block and the prosthetic limb to reduce the stress on the prosthetic limb, and increasing suspension ability.

It should be noted that similar principles enunciated above can be applied to other areas such as, for example, ski boots, helmets, and the like. In such cases, a gel bag equipped with a one-way valve may also be used to capture the specific shape of a user's head or foot, for example, so as to provide increase adjustability, support and comfort.

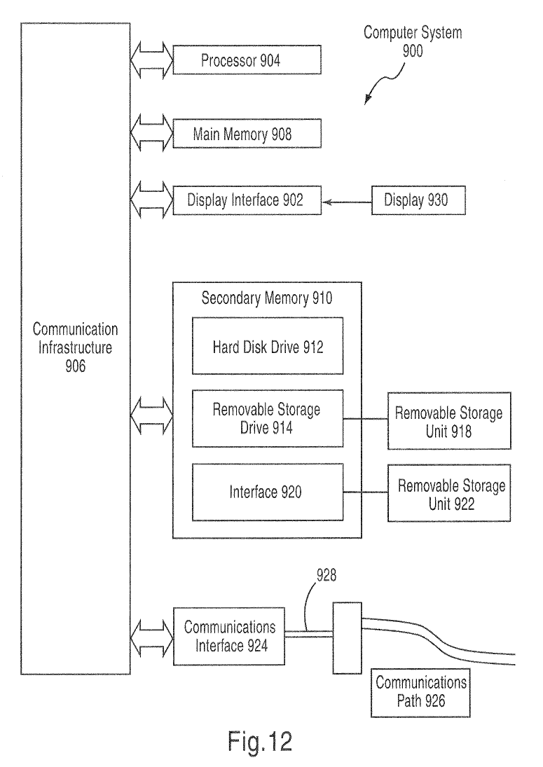

It should be noted that many aspects of the above-discussed method can be implemented using a computer and software. FIG. 12 presents an exemplary system diagram of various hardware components and other features, for use in accordance with an aspect of the present invention. Aspects of some variations of the present invention may be implemented using hardware, software, or a combination thereof and may be implemented in one or more computer systems or other processing systems. In one aspect, the invention is directed toward one or more computer systems capable of carrying out the functionality described herein. An example of such a computer system 900 is shown in FIG. 12.

Computer system 900 includes one or more processors, such as processor 904. The processor 904 is connected to a communication infrastructure 906 (e.g., a communications bus, cross-over bar, or network). Various software aspects are described in terms of this exemplary computer system. After reading this description, it will become apparent to a person skilled in the relevant art(s) how to implement the invention using other computer systems and/or architectures.

Computer system 900 can include a display interface 902 that forwards graphics, text, and other data from the communication infrastructure 906 (or from a frame buffer not shown) for display on a display unit 930. Computer system 900 also includes a main memory 908, preferably random access memory (RAM), and may also include a secondary memory 910. The secondary memory 910 may include, for example, a hard disk drive 912 and/or a removable storage drive 914, representing a floppy disk drive, a magnetic tape drive, an optical disk drive, etc. The removable storage drive 914 reads from and/or writes to a removable storage unit 918 in a well-known manner. Removable storage unit 918, represents a floppy disk, magnetic tape, optical disk, etc., which is read by and written to removable storage drive 914. As will be appreciated, the removable storage unit 918 includes a computer usable storage medium having stored therein computer software and/or data. In alternative aspects, secondary memory 910 may include other similar devices for allowing computer programs or other instructions to be loaded into computer system 900. Such devices may include, for example, a removable storage unit 922 and an interface 920. Examples of such may include a program cartridge and cartridge interface (such as that found in video game devices), a removable memory chip (such as an erasable programmable read only memory (EPROM), or programmable read only memory (PROM)) and associated socket, and other removable storage units 922 and interfaces 920, which allow software and data to be transferred from the removable storage unit 922 to computer system 900.

Computer system 900 may also include a communications interface 924. Communications interface 924 allows software and data to be transferred between computer system 900 and external devices. Examples of communications interface 924 may include a modem, a network interface (such as an Ethernet card), a communications port, a Personal Computer Memory Card International Association (PCMCIA) slot and card, etc. Software and data transferred via communications interface 924 are in the form of signals 928, which may be electronic, electromagnetic, optical or other signals capable of being received by communications interface 924. These signals 928 are provided to communications interface 924 via a communications path (e.g., channel) 926. This path 926 carries signals 928 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 980, a hard disk installed in hard disk drive 970, and signals 928. These computer program products provide software to the computer system 900. The invention is directed to such computer program products.

Computer programs (also referred to as computer control logic) are stored in main memory 908 and/or secondary memory 910. Computer programs may also be received via communications interface 924. Such computer programs, when executed, enable the computer system 900 to perform the features of the present invention, as discussed herein. In particular, the computer programs, when executed, enable the processor 910 to perform the features of the present invention. Accordingly, such computer programs represent controllers of the computer system 900.

In an aspect where the invention is implemented using software, the software may be stored in a computer program product and loaded into computer system 900 using removable storage drive 914, hard drive 912, or communications interface 920. The control logic (software), when executed by the processor 904, causes the processor 904 to perform the functions of the invention as described herein. In another aspect, the invention is implemented primarily in hardware using, for example, hardware components, such as application specific integrated circuits (ASICs). Implementation of the hardware state machine so as to perform the functions described herein will be apparent to persons skilled in the relevant art(s).

In yet another aspect, the invention is implemented using a combination of both hardware and software.

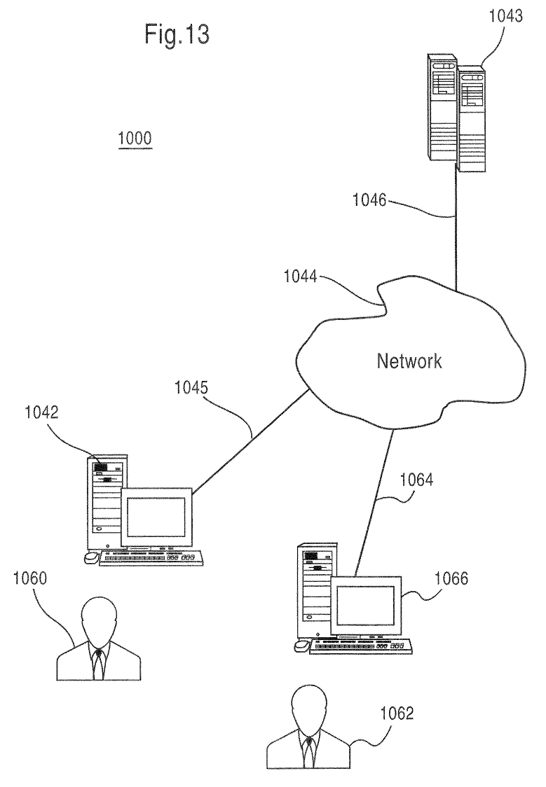

FIG. 13 is a block diagram of various exemplary system components, for use in accordance with some variations of the present invention. FIG. 13 shows a computer system 1000 usable in accordance with the present invention. The computer system 1000 includes one or more accessors 1060, 1062 (also referred to interchangeably herein as one or more “users”) and one or more terminals 1042, 1066. In one aspect, data for use in accordance with aspects of the present invention is, for example, input and/or accessed by accessors 1060, 1064 via terminals 1042, 1066, such as personal computers (PCs), minicomputers, mainframe computers, microcomputers, telephonic devices, or wireless devices, such as personal digital assistants (“PDAs”) or a hand-held wireless devices coupled to a server 1043, such as a PC, minicomputer, mainframe computer, microcomputer, or other device having a processor and a repository for data and/or connection to a repository for data, via, for example, a network 1044, such as the Internet or an intranet, and couplings 1045, 1046, 1064. The couplings 1045, 1046, 1064 include, for example, wired, wireless, or fiberoptic links. In another aspect, the method and system of the present invention may operate in a stand-alone environment, such as on a single terminal.

While this invention has been described in conjunction with the exemplary aspects outlined above, various alternatives, modifications, variations, improvements, and/or substantial equivalents, whether known or that are or may be presently unforeseen, may become apparent to those having at least ordinary skill in the art. Accordingly, the exemplary aspects of the invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents.

What is claimed is:

1. A vacuum-based alignment method for manufacturing a prosthetic device for a residual limb of a patient, the method comprising:
adjusting the residual limb in a malleable bag;
performing alignment of the residual limb while the residual limb is in the malleable bag;
generating a mold of the residual limb from an imprint of the residual limb in the malleable bag; and
forming the prosthetic device by using the mold of the residual limb.

2. The method of claim 1, wherein adjusting the residual limb comprises:
pressing the residual limb against the malleable bag contemporaneously with injecting air into the malleable bag to fit the residual limb within the malleable bag; and
hardening the malleable bag by removing air from the malleable bag once the residual limb is adjusted within the malleable bag.

3. The method of claim 2, wherein removing air from the malleable bag is performed using at least one selected form the group consisting of a manual pump and human lung capacity.

4. The method of claim 1, wherein performing the alignment comprises at least one of:
performing static alignment; and
performing dynamic alignment.

5. The method of claim 4, wherein performing the static alignment comprises:
having the patient stand up while the residual limb is within the malleable bag; and
adjusting the malleable bag to account for soft tissue pressure.

6. The method of claim 4, wherein performing the dynamic alignment comprises:
having the patient walk while the residual limb is within the malleable bag;
adjusting the malleable bag to account for soft tissue pressure; and
hardening the malleable bag by removing air from the malleable bag.

7. The method of claim 1, wherein generating a mold of the residual limb comprises:
removing the malleable bag from the residual limb to leave an impression within the malleable bag; and
inserting molding material in the impression for use in producing a mold of the residual limb.

8. The method of claim 1, wherein forming the prosthetic device comprises:
attaching the mold to a vertical jig, wherein the vertical jig comprises an ankle block located vertically below the solid mold;
connecting the attached mold to the ankle block via connectors, wherein the connectors are attached to the mold at one or more attachment points;
emplacing a liner over the mold, the connectors and the ankle block; and
vacuum-forming the liner so as to produce the prosthetic device.

9. The method of claim 8, wherein the connectors comprise strings.

10. The method of claim 8, wherein the one or more attachment points are located so as to ensure an adequate distribution of the weight of the patient.

11. The method of claim 8, wherein emplacing the liner comprises:
softening the liner;
draping the liner over the mold, the connectors and the ankle block; and
sealing the liner over the mold, the connectors and the ankle block.

12. The method of claim 11, wherein softening the liner comprises subjecting the liner to heat.

13. The method of claim 12, further comprising solidifying the liner by cooling the liner so as to form the prosthesis.

14. The method of claim 8, wherein forming the prosthetic device comprises removing the mold from the liner.

15. A vacuum-based impression and alignment system, comprising:
a malleable bag coupled to a connector; and
a reversible one-way valve connected to the malleable bag via the connector; wherein a tube is connected to the reversible one-way valve.

16. The vacuum-based system of claim 15, further comprising a pump to transfer air to and from the malleable bag via the reversible one-way valve.

17. The vacuum-based system of claim 16, wherein the pump is a manual pump.

18. The vacuum-based system of claim 15 wherein the connector is connected to a simulated foot on a side opposite to the malleable bag.

19. A system for manufacturing a vacuum-based alignment prosthetic device for a residual limb of a patient, the system comprising:
means for adjusting the residual limb in a malleable bag;
means for performing alignment of the residual limb in the malleable bag;
means for generating a solid impression of the residual limb from an imprint of the residual limb in the malleable bag; and
means for forming the prosthetic device by using of the solid impression of the residual limb.

20. The vacuum-based alignment method of claim 11, wherein the liner comprises a plastic material.

21. The vacuum-based alignment method of claim 1, wherein the malleable bag comprises a gel bag.
